SEQUENCE LISTING (12 sequences)

SEQ ID NO: 1 - DNA, 42 bp, synthetic construct
  misc_feature 1..42: note = primer 1
  source 1..42: mol_type = other DNA
cgacatggctacgatccgacctacacgacgctcttccgatct

SEQ ID NO: 2 - DNA, 28 bp, synthetic construct
  misc_feature 1..28: note = primer 2
  source 1..28: mol_type = other DNA
agttgnaagcagtggtatcaacgcagag

SEQ ID NO: 3 - DNA, 28 bp, synthetic construct
  misc_feature 1..28: note = primer 3
  source 1..28: mol_type = other DNA
acaacnctacacgacgctcttccgatct

SEQ ID NO: 4 - DNA, 28 bp, synthetic construct
  misc_feature 1..28: note = primer 4
  source 1..28: mol_type = other DNA
acttcnaagcagtggtatcaacgcagag

SEQ ID NO: 5 - DNA, 28 bp, synthetic construct
  misc_feature 1..28: note = primer 5
  source 1..28: mol_type = other DNA
agaagnctacacgacgctcttccgatct

SEQ ID NO: 6 - DNA, 28 bp, synthetic construct
  misc_feature 1..28: note = primer 6
  source 1..28: mol_type = other DNA
atggtnaagcagtggtatcaacgcagag

SEQ ID NO: 7 - DNA, 28 bp, synthetic construct
  misc_feature 1..28: note = primer 7
  source 1..28: mol_type = other DNA
aaccanctacacgacgctcttccgatct

SEQ ID NO: 8 - DNA, 42 bp, synthetic construct
  misc_feature 1..42: note = primer 8
  source 1..42: mol_type = other DNA
cgacatggctacgatccgacaagcagtggtatcaacgcagag

SEQ ID NO: 9 - DNA, 20 bp, synthetic construct
  source 1..20: mol_type = other DNA
  misc_feature 1..20: note = seletion primer
cgacatggctacgatccgac

SEQ ID NO: 10 - DNA, 78 bp, synthetic construct
  source 1..78: mol_type = other DNA
  misc_feature 1..78: note = PB barcoded adapter sequence
cgcactctgatatgtgatctctctcttttcctcctcctccgttgttgttgttgagagagatcacatatcagagtgcgt

SEQ ID NO: 11 - DNA, 22 bp, synthetic construct
  source 1..22: mol_type = other DNA
  misc_feature 1..22: note = Forward primer
ctacacgacgctcttccgatct

SEQ ID NO: 12 - DNA, 22 bp, synthetic construct
  source 1..22: mol_type = other DNA
  misc_feature 1..22: note = Reverse primer
aagcagtggtatcaacgcagag

(19) United States
(12) Patent Application Publication
(10) Pub. No.: US 2024/0279729 A1
(43) Pub. Date: Aug. 22, 2024

(54) AMPLIFICATION PRIMER DESIGN AND LIGATION METHOD FOR DNA MOLECULES

(71) Applicant: BGI TECH SOLUTIONS CO., LTD., Shenzhen (CN)

(72) Inventors: Zhichao CHEN, Shenzhen (CN); Chong Tang, Shenzhen (CN); Fengying Ruan, Shenzhen (CN); Yaning Li, Shenzhen (CN); Mei Guo, Shenzhen (CN)

(73) Assignee: BEI TECH SOLUTIONS CO., LTD., Shenzhen (CN)

(21) Appl. No.: 18/171,810

(22) Filed: Feb. 21, 2023

(51) Int. Cl. C12Q 1/686; C12N 15/10; C12Q 1/6806

(52) U.S. Cl. CPC C12Q 1/686; C12N 15/1093; C12Q 1/6806; C12Q 2600/16

(57) ABSTRACT

The present disclosure discloses an amplification primer design and ligation method for DNA molecules, particularly a library preparation method for increasing the throughput of data available for sequencing Pacbio amplicons, which includes designing different primer pairs for the DNA molecule and performing PCR amplification on the template DNA using the different primer pairs respectively in different amplification systems. In the present disclosure, shorter PCR products are ligated to obtain a longer library, and the ligated library is used for sequencing, which can greatly improve data utilization while ensuring data quality.

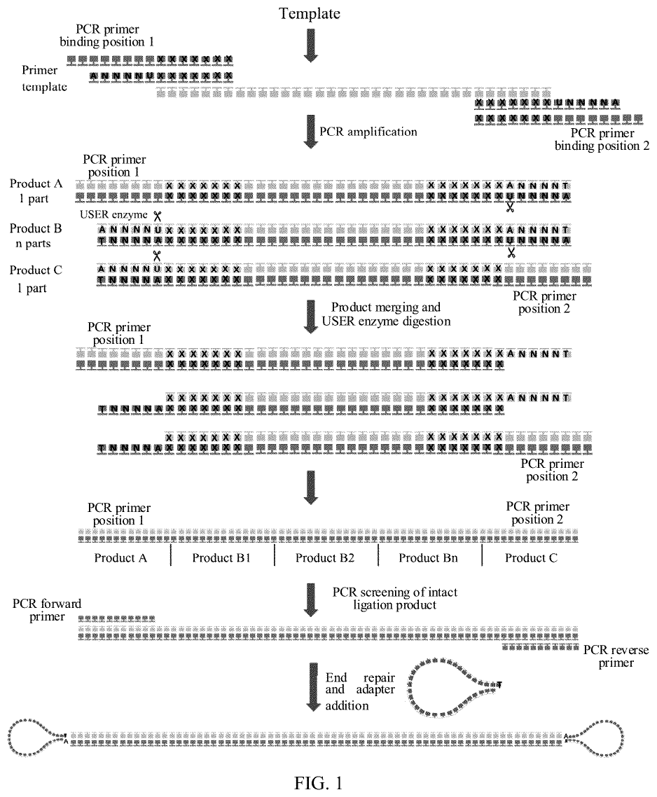

### REFERENCES TO AN ELECTRONIC SEQUENCE LISTING

The contents of the electronic sequence listing POI222897USSequencelisting.xml; Size 16 KB bytes; Date of Creation: Feb. 15, 2023) is hereby incorporated by reference in its entirety.

### TECHNICAL FIELD

The present disclosure relates to the field of biological sequencing. It particularly relates to an amplification primer design and ligation method for DNA molecules, particularly a library preparation method for increasing the throughput of Pacbio amplicon sequencing data.

### BACKGROUND

In some sequencing applications, library preparation requires amplification, and the inserted fragment is long, so long-reads sequencing needs to be used, such as full-length 16S rDNA sequencing, 18S rDNA sequencing, ITS sequencing, target gene sequencing, full-length transcriptome sequencing, and targeted full-length transcriptome sequencing etc. In the Pacbio long-reads sequencing platform, after several versions of the development of the sequencing reagents, the sequencing length has been improved to 60 k or higher. Since the Pacbio sequencing chip can only read one molecule per well, the DNA library molecules formed by amplification products are repeatedly sequenced in the sequencing wells, resulting in redundancy and waste of the enzyme reads.

At present, in different applications, the conventional process of preparing a Pacbio library after obtaining an amplification product includes, sequentially, DNA damage repair of the amplification product, end repair, magnetic bead purification, adapter ligation, removal of incomplete library, and magnetic bead purification.

The routine library preparation process includes: performing DNA damage repair on the PCR product obtained by amplification (e.g., microbial genome amplification using 16S rDNA full-length primers), adding a sequencing adapter, removing the incomplete library using a digestive enzyme, and performing purification to a sequencing library.

Generally, the length of the amplicon and the average length of the full-length transcriptome are relatively short, the length of the amplicon 16S rDNA is about 1.5 k, the length of the 18S rDNA is about 1.8 k, the average length of the full-length transcriptome is around 2 k, and the length of the immune cell TCR/BCR full-length transcript is about 1 k to 1.8 k. The average length of the libraries obtained by conventional library preparation is less than 2 k. The reads of the Pacbio sequencing platform can reach 60 k or higher, i.e., the number of sequencing cycles of a single library molecule reaches 30 cycles or more. According to statistics, 6 sequencing cycles are already sufficient for accurate sequence analysis. Therefore, the excessive sequencing data is a large data redundancy, that is to say, the existing library preparation methods cannot fully use the advantages of 60 kb reads of the Pacbio platform.

### SUMMARY

The technical problem to be solved by the present disclosure is to provide an amplification primer design and ligation method for DNA molecules to overcome the defects in the related art that the existing method for preparing a library of amplification products cannot make full use of the advantages of long reads of the Pacbio platform, and to design a scheme for library preparation by end-to-end ligation, which connects the PCR products end-to-end via cohesive ends, increasing the average length of the library and obtaining more effective data after sequencing.

The present disclosure mainly solves the above technical problem by the following technical solutions.

The present disclosure provides a set of PCR primer pairs. An association between different primer pairs of the set of PCR primer pairs satisfies: for two different primer pairs, a forward primer of one primer pair differs from a reverse primer of the other primer pair in n bases at a 5′ end, wherein a first base in the n bases is a base A, and an n-th base in the n bases is a base dU, where n is an integer greater than or equal to 4. A forward primer of a first primer pair and a reverse primer of the last primer pair do not have the characteristic association.

Preferably, n is in a range from 4 to 30, and more preferably, n is 6.

The present disclosure also provides an amplification method of a DNA molecule. The amplification method includes: designing different primer pairs for the DNA molecule, and performing PCR amplification on a template DNA using the different primer pairs respectively in different amplification systems, A characteristic association between the different primer pairs satisfies: for two different primer pairs, a forward primer of one primer pair differs from a reverse primer of the other primer pair in n bases at a 5′ end, wherein a first base in the n bases is a base A, and an n-th base in the n bases is a base dU, where n is an integer greater than or equal to 4. A forward primer of a first primer pair and a reverse primer of a last primer pair do not have the characteristic association.

Preferably, n is in a range from 4 to 30, and more preferably, n is 6.

The amplification method of the present disclosure preferably further includes, after performing the PCR amplification, ligating amplification products obtained from the different amplification systems.

Preferably, said ligating is performed by removing bases dU of the amplification products to create cohesive ends.

Preferably, the bases dU are removed by digestion using Uracil-DNA Glycosylase and Endonuclease VIII in USER enzyme.

The present disclosure also provides a library preparation method for increasing a throughput of Pacbio amplicon sequencing data, including the amplification method mentioned above.

In a specific embodiment of the present disclosure, before PCR is performed on the template, the PCR system is divided into n tubes, different PCR primers are added to different tubes, and the forward primers or reverse primers of different primer pairs (except the forward primer of tube 1 and the reverse primer of tube n) differs only in several linking sequences at the 5′ ends, wherein the first base at the 5′ end of the linking sequence is a base A and the last base is a base dU (for example, there is a linking sequence containing 6 bases at the 5′ end, the first base is a base A, and the 6th base is a base dU). The base dU can be removed by digestion using USER enzyme Uracil-DNA Glycosylase (UDG) and Endonuclease VIII to generate a cohesive end, the series of cohesive ends is characterized in that product A is ligated only to product B1 with complementary ligation, product B1 is ligated only to product A and product B2, product B(n-1) is ligated only to product B(n-2) and product Bn, and product Bn is ligated only to product B(n-1) and product C in complementary ligation. The products with cohesive ends are ligated using DNA ligase, and due to the specific characteristics of the sequences of the cohesive ends, different products are sequentially ligated end-to-end.

Since the ligation efficiency of ligase is not 100%, and some of the ligation products contain nicks, it is necessary to perform PCR amplification to screen out the intact ligation product; the forward primer of tube 1 and the reverse primer of tube n do not have a base dU at the 5′ end and can be used as a primer binding position for the screening PCR. A standard dumbbell-shaped library can be obtained by adding sequencing adapters to the PCR product.

The present disclosure can fully use the super-long reads of Pacbio sequencing by designing a set of dU-containing primers to enable end-to-end ligation of a fixed number of amplified fragments during library preparation of Pacbio amplicon.

The present disclosure applies to using Pacbio sequencing to obtain sequence information for amplicons, such as full-length transcripts, 16S, 18S and other amplicons of gene fragments of interest.

In the present disclosure, a library preparation process is redesigned based on the Pacbio official full-length transcriptome library preparation process, and the length of the library is increased through ligation of the transcript PCR products, increasing the utilization of sequencing data.

The present disclosure further provides a library preparation method for increasing a throughput of Pacbio amplicon sequencing data. The library preparation method includes the amplification method as described above.

The present disclosure further provides a method of sequencing a full-length transcriptome. The method includes the amplification method as described above.

The present disclosure further provides the use of the PCR primer pairs as described above in the amplification of DNA molecules, in increasing the throughput of Pacbio amplicon sequencing data, or in full-length transcriptome sequencing.

The present disclosure further provides a set of primer pairs for the amplification of TCR/BCR cDNA, and sequences of the primer pairs are set forth in SEQ ID NOs: 1-8.

The preferred embodiments of the present disclosure can be obtained by the combination of the above-mentioned preferred conditions, provided that the combination does not contradict common knowledge in the art.

The reagents and starting materials used in the present disclosure are commercially available.

The present disclosure has the following beneficial and positive effects.

In the present disclosure, shorter PCR products are ligated to obtain a longer library, and the library obtained through the ligation is used for sequencing, which can greatly improve data utilization while ensuring data quality. In this way, the throughput is increased up to 3.06 times.

### BRIEF DESCRIPTION OF DRAWINGS

FIGURE is a schematic diagram of the principle of library preparation by end-to-end ligation.

### DESCRIPTION OF EMBODIMENTS

The present disclosure is further illustrated with the following examples but is not limited to the scope of the described examples. The experimental methods for which specific conditions are not specified in the following examples are conducted according to conventional methods and conditions or according to commercial specifications.

The ligation-based library preparation technique of the present disclosure and a conventional library preparation method were used respectively for the preparation of a library of the full-length TCR/BCR cDNA (known in the art) amplification products obtained from the 10× Genomics platform were prepared into a library (see the figure for the principle of library preparation), and the prepared library was then sequenced.

### Example 1 Library Preparation Using the Ligation-Based Library Preparation Technique of the Present Disclosure

### 1. PCR Amplification

### 1.1 Second Strand Synthesis

Taking 20 ng of the TCR/BCR captured PCR product to prepare the following PCR system:

| Reagent name | Dosage | |
|---|---|---|
| Captured PCR product | 20 | ng |
| KAPA HiFi HotStart Uracil + ReadyMix (2×) | 50 | μl |

| | |
|---|---|
| Nuclease-free water (NF water) | Making up |
| | to 94 μl |

Dividing the PCR system into 4 tubes, adding the following 4 pairs of primers in each tube, each primer (10 mM) added in an amount of 0.75 μl:

| | | Primer sequence 5′-3′ | SEQ |
|---|---|---|---|
| | Primer | (N in the sequence | ID |
| | name | represents a base dU) | NO: |
| Tube 1 | 4Link- | CGACATGGCTACGATCCGACCTACA | 1 |
| | Forward1 | CGACGCTCTTCCGATCT | |
| | 4Link- | AGTTGNAAGCAGTGGTATCAACGC | 2 |
| | Reverse1 | AGAG | |
| Tube 2 | 4Link- | ACAACNCTACACGACGCTCTTCCG | 3 |
| | Forward2 | ATCT | |
| | 4Link- | ACTTCNAAGCAGTGGTATCAACGC | 4 |
| | Reverse2 | AGAG | |
| Tube 3 | 4Link- | AGAAGNCTACACGACGCTCTTCCG | 5 |
| | Forward3 | ATCT | |
| | 4Link- | ATGGTNAAGCAGTGGTATCAACGC | 6 |
| | Reverse3 | AGAG | |
| Tube 4 | 4Link- | AACCANCTACACGACGCTCTTCCG | 7 |
| | Forward4 | ATCT | |
| | 4Link- | CGACATGGCTACGATCCGACAAGC | 8 |
| | Reverse4 | AGTGGTATCAACGCAGAG | |

The PCR reaction procedure is as follows:

| | | |
|---|---|---|
| Temperature | Reaction time | Number of cycles |

| | | | | |
|---|---|---|---|---|
| 95° | C. | 2 | min | 1 |
| 98° | C. | 20 | s | 7 |
| 63° | C. | 15 | s | |
| 72° | C. | 2 | min | |
| 72° | C. | 5 | min | 1 |

| | | | |
|---|---|---|---|
| 4° | C. | ∞ | 1 |

### 1.2 Sample Purification

1) Performing Qubit quantification on each of the four tubes of PCR products and taking equal amounts from the four tubes for mixing to obtain a PCR mixture.
2) Adding AMPure XP magnetic beads (1× volume) into a 1.5 mL centrifuge tube filled with the PCR mixture, mixing well, and performing instantaneous centrifugation. Incubating at room temperature for 5 min.
3) Transferring and placing the centrifuge tube onto a magnetic rack, and after standing still until clear, removing the supernatant by aspiration.
4) Adding 300 μL of 80% ethanol and discarding the supernatant after standing still for 30 s.
5) Repeating the previous step once, uncapping, and air drying for 5 min to remove residual ethanol.
6) Re-dissolving with 19 μL of elution buffer and placing on ThemoMixer, 20° C., 2,000 rpm, 10 min.
7) At the end of 10 min, taking out the centrifuge tube, transiently centrifuging for 2 s, placing on a magnetic rack, standing still until clear, aspirating the supernatant and recovering in another labeled 1.5 mL centrifuge tube.
8) Taking 1 μL of the purified sample and diluting by 5 times, taking 1 μL of the diluted solution for Qubit detection, and performing 2100 quality inspection on the remaining diluted sample.

### 2. End-to-End Sequential Ligation

Taking the purified PCR product to prepare the following system:

| Reagents | Volume/μL |
|---|---|
| Purified PCR product | 17 |
| 10 × T4 DNA ligase buffer | 2 |
| USER Enzyme (1 unit) | 1 |

After mixing well, reacting at 37° C. for 20 min. Adding 1 μL of T4 DNA ligase (NEB), mixing well, and reacting for 1 h at 16° C.

Adding 80 μL of water to make up to 100 μL. Adding Ampure XP magnetic beads (0.4× volume, 40 μL) into the tube for purification, and finally redissolving in 20 μL elution buffer. The concentration of the purified product was determined by Qubit dsDNA HS Assay kit.

### 3. PCR Amplification of Ligated Product

Taking 100 ng of the purified ligation product to prepare the following PCR system:

| Reagent name | Amount | |
|---|---|---|
| Ligation product | 100 | ng |
| KAPA HiFi HotStart ReadyMix (2×) | 50 | μL |
| Primer Selection primer (10 mM) | 6 | μL |

| | |
|---|---|
| Nuclease-free water (NF water) | Making up |
| | to 100 μL |

Selection Primer: 5′ PHO-CGACATGGCTACGATCCGAC-3′ (SEQ ID NO: 9) was used to screen PCR primers of the effective ligation products.

The PCR reaction procedure is as follows:

| | | |
|---|---|---|
| Temperature | Reaction time | Number of cycles |

| | | | | |
|---|---|---|---|---|
| 95° | C. | 2 | min | 1 |
| 98° | C. | 20 | s | 8-9 |
| 65° | C. | 15 | s | |
| 72° | C. | 5 | min | |
| 72° | C. | 5 | min | 1 |

| | | | |
|---|---|---|---|
| 4° | C. | ∞ | 1 |

Adding Ampure XP magnetic beads (0.4× volume, 40 μL) into the tube for purification, and finally redissolving in 27 μL elution buffer.

### 4. End Repair and Adapter Addition (Using NEBNext® Ultra™ II DNA Library Prep Kit for Illumina)

### 4.1 End Repair

Taking the purified ligation product to prepare the following system:

| Reagents | Volume/μL |
|---|---|
| Purified product of previous step | 25 |
| NEBNext Ultra II End Prep Reaction Buffer | 3.5 |
| NEBNext Ultra II End Prep Enzyme Mix | 1.5 |

After mixing well, reacting at 20° C. for 30 min, reacting at 65° C. for 30 min, and holding at 4° C.

### 4.2 Sequencing Adapter Addition

Adding the following system to the product of the previous step:

| Reagents | Volume/μL |
|---|---|
| NEBNext Ultra II Ligation Master Mix | 15 |
| NEBNext Ligation Enhancer | 0.5 |
| Adapter (PB barcoded adapter) | 1.5 |

Note: the PB barcoded adapter sequence is: 5′-/5Phos/ (16 bp barcode)ATCTCTCTCTTTTCCTCCTCCTCCGTTGTTGTTGTTGAGAGAGAT(16 bp barcode)T-3′, where the 16 bp barcode is not fixed, and the two 16 bp barcodes at the beginning and the end of the adapter sequence are complementary with each other. For example, the PB barcoded adapter sequence is: 5′-/5Phos/CGCACTCTGATATGTGATCTCTCTCTTTTCCTCCTCCTCCGTTGTTGTTGTTGAG AGAGATCACATATCAGAGTGCGT-3′ (SEQ ID NO: 10).

After mixing well, reacting at 20° C. for 30 min.

### 5. Enzyme Digestion and Magnetic Bead Purification

1) Adding the following mix to the product of the previous step:

| Reagents | Volume/μL |
|---|---|
| Reaction buffer (NEBuffer I, 10×) | 10 |
| Exonuclease I (Exonuclease I, 20 U/μL, NEB) | 2 |
| Exonuclease III (Exonuclease III, 100 U/μL, NEB) | 2 |
| Nuclease-free water (NF water) | 39 |

After mixing well, reacting at 37° C. for 1 h.
2) Adding AMpure XP magnetic beads (0.4× volume, 40 μL) into the 1.5 mL centrifuge tube containing the product of the enzymatic digestion reaction for purification, and finally redissolving in 15 μL of elution buffer.
3) Taking 1 μL of the purified sample and diluting by 5 times, taking 1 μL of the diluted sample for Qubit detection, and performing 2100 quality inspection on the remaining diluted sample.

### 6. Sequencing

Preparing and sequencing were performed according to the corresponding instructions by using the Diffusion loading protocol provided by Pacbio.

### Example 2 Library Preparation Using the Conventional Library Preparation Technique

### 1. PCR Amplification

Taking 20 ng of the TCR/BCR captured PCR product to prepare the following PCR system:

| Reagent name | Dosage | |
|---|---|---|
| Captured PCR product | 20 | ng |
| KAPA HiFi HotStart ReadyMix (2×) | 50 | μL |
| Forward primer (10 mM) | 3 | μL |
| Reverse primer (10 mM) | 3 | μL |

| | |
|---|---|
| Nuclease-free water (NF water) | Making up |
| | to 100 μL |

Primer information is as follows:

| | | SEQ |
|---|---|---|
| Primer | Primer sequence | ID |
| name | 5′-3′ | NO: |
| Forward primer | PHO-CTACACGACGCTCTTCCGATCT | 11 |
| Reverse primer | PHO-AAGCAGTGGTATCAACGCAGAG | 12 |

The PCR reaction procedure is as follows:

| | | |
|---|---|---|
| Temperature | Reaction time | Number of cycles |

| | | | | |
|---|---|---|---|---|
| 95° | C. | 2 | min | 1 |
| 98° | C. | 20 | s | 7 |
| 63° | C. | 15 | s | |
| 72° | C. | 2 | min | |
| 72° | C. | 5 | min | 1 |

| | | | |
|---|---|---|---|
| 4° | C. | ∞ | 1 |

Detecting the concentration of the PCR product with Qubit dsDNA HS Assay kit, and the concentration shall be greater than 20 ng/uL. Adding Ampure XP magnetic beads (0.8× volume, 80 μL) into the tube for purification, and finally redissolving in 27 μL of elution buffer.

### 2. End Repair and Adapter Addition (Using NEBNext® Ultra™ II DNA Library Prep Kit for Illumina)

### 2.1 End Repair

Taking the purified ligation product to prepare the following system:

| Reagents | Volume/μL |
|---|---|
| Purified product of previous step | 25 |
| NEBNext Ultra II End Prep Reaction Buffer | 3.5 |
| NEBNext Ultra II End Prep Enzyme Mix | 1.5 |

After mixing well, reacting at 20° C. for 30 min, reacting at 65° C. for 30 min, and holding at 4° C.

### 2.2 Sequencing Adapter Addition

Adding the following system to the product of the previous step:

| Reagents | Volume/μL |
|---|---|
| NEBNext Ultra II Ligation Master Mix | 15 |
| NEBNext Ligation Enhancer | 0.5 |
| Adapter (PB barcoded adapter) | 1.5 |

Note: the sequence of the adapter is the same as the adapter sequence used in 4.2 of Example 1.

After mixing well, reacting at 20° C. for 30 min.

### 3. Enzyme Digestion and Magnetic Bead Purification

1) Adding the following mix to the product of the previous step:

| Reagents | Volume/μL |
|---|---|
| Reaction buffer (NEBuffer I, 10×) | 10 |
| Exonuclease I (Exonuclease I, 20 U/μL, NEB) | 2 |
| Exonuclease III (Exonuclease III, 100 U/μL, NEB) | 2 |
| Nuclease-free water (NF water) | 39 |

After mixing well, reacting at 37° C. for 1 h.
2) Adding Ampure XP magnetic beads (0.8× volume, 80 μL) into the 1.5 mL centrifuge tube containing the product of the enzymatic digestion reaction for purification, and finally redissolving in 15 μL of elution buffer.
3) Taking 1 μL the purified sample and diluting by 5 times, taking 1 μL of the diluted sample for Qubit detection, and performing 2100 quality inspection on the remaining diluted sample.

### 4. Sequencing

Preparing and sequencing were performed using the Diffusion loading protocol provided by Pacbio according to the corresponding instructions.

### Test Results

TABLE 1
Data analysis results

| | | | | Reads/ | Throughput |
|---|---|---|---|---|---|
| | Number | Number | Reads/ | Million | enhancement |
| Library | of CCSs | of Reads | CCS | CCS | times |
| Ligation- | 4858856 | 11253405 | 2.32 | 2316060 | 3.06 |
| based | | | | | |
| method | | | | | |
| Conventional | 5258552 | 3978197 | 0.76 | 756519 | |

Where the number of CCSs indicates the number of circular consensus sequences, and one CCS was measured from one sequencing well.

Here, the number of Reads represents the number of effective transcripts obtained from CCSs through data analysis.

Since each CCS only contains information of one transcript in the data obtained by conventional library preparation, the number of the transcript Reads after data filtering is lower than the number of CCSs; however, in the data obtained from the library preparation by the ligation-based method of the present disclosure, each CCS contains information of multiple transcripts, so the obtained transcript Reads are more than the CCSs (Table 1). The present disclosure can greatly increase the number of transcripts detected.

1. A set of PCR primer pairs, a characteristic association between different primer pairs of which satisfying:
for two different primer pairs, a forward primer of one primer pair differs from a reverse primer of the other primer pair in n bases at a 5′ end, wherein a first base in the n bases is a base A, and an n-th base in the n bases is a base dU, where n is an integer greater than or equal to 4.

2. The set of PCR primer pairs according to claim 1, wherein n is in a range from 4 to 30, and preferably, n is 6.

3. An amplification method of a DNA molecule, comprising:
designing different primer pairs for the DNA molecule; and
performing PCR amplification on a template DNA using the different primer pairs respectively in different amplification systems,
wherein a characteristic association between the different primer pairs satisfies:
for two different primer pairs, a forward primer of one primer pair differs from a reverse primer of the other primer pair in n bases at a 5′ end, wherein a first base in the n bases is a base A, and an n-th base in the n bases is a base dU, where n is an integer greater than or equal to 4.

4. The amplification method according to claim 3, wherein n is in a range from 4 to 30.

5. The amplification method according to claim 3, wherein n is 6.

6. The amplification method according to claim 3, further comprising, after performing the PCR amplification:
ligating amplification products obtained from the different amplification systems.

7. The amplification method according to claim 6, wherein said ligating is performed by removing bases dU of the amplification products to create cohesive ends.

8. The amplification method according to claim 7, wherein the bases dU are removed by digestion using Uracil-DNA Glycosylase and Endonuclease VIII in USER enzyme.

9. A library preparation method for increasing a throughput of Pacbio amplicon sequencing data, comprising the amplification method according to claim 3.

10. The library preparation method according to claim 9, wherein n is in a range from 4 to 30.

11. The library preparation method according to claim 9, wherein the amplification method further comprises, after performing the PCR amplification:
ligating amplification products obtained from the different amplification systems.

12. The library preparation method according to claim 11, wherein said ligating is performed by removing bases dU of the amplification products to create cohesive ends.

13. The library preparation method according to claim 12, wherein the bases dU are removed by digestion using Uracil-DNA Glycosylase and Endonuclease VIII in USER enzyme.

14. A method for sequencing a full-length transcriptome, comprising the amplification method according to claim 3.

15. The method according to claim 14, wherein n is in a range from 4 to 30.

16. The method according to claim 14, wherein the amplification method further comprises, after performing the PCR amplification:
ligating amplification products obtained from the different amplification systems.

17. The method according to claim 16, wherein said ligating is performed by removing bases dU of the amplification products to create cohesive ends.

18. The method according to claim 17, wherein the bases dU are removed by digestion using Uracil-DNA Glycosylase and Endonuclease VIII in USER enzyme.

19. A set of primer pairs for amplification of TCR/BCR cDNA, sequences of the primer pairs being set forth in SEQ ID NOs: 1-8.

20. The set of PCR primer pairs according to claim 1, wherein a forward primer of a first primer pair and a reverse primer of a last primer pair do not have the characteristic association.
